SEQUENCE LISTING (6 sequences)

SEQ ID NO: 1 - DNA, 20 bp, Artificial sequence
  Synthetic Primer Gck Forward
ttgctctaaggggaccagaa

SEQ ID NO: 2 - DNA, 20 bp, Artificial sequence
  Synthetic Primer Gck Reverse
ggaacgagggagagaaggac

SEQ ID NO: 3 - DNA, 20 bp, Artificial sequence
  Synthetic Primer G6pc Forward
ctaccttgcggctcactttc

SEQ ID NO: 4 - DNA, 20 bp, Artificial sequence
  Synthetic Primer G6pc Reverse
atccaagtgcgaaaccaaac

SEQ ID NO: 5 - DNA, 21 bp, Artificial sequence
  Synthetic Primer Pklr Forward
ctggaacacctctgccttctg

SEQ ID NO: 6 - DNA, 21 bp, Artificial sequence
  Synthetic Primer Pklr Reverse
cacaatttccacctccgactc

(19) United States
(12) Patent Application Publication
(10) Pub. No.: US 2020/0188394 A1
(43) Pub. Date: Jun. 18, 2020

(54) TREATMENT OF LIVER DISEASE

(71) Applicant: UNIVERSITY OF NEWCASTLE UPON TYNE, Tyne and Wear (GB)

(72) Inventor: Loranne AGIUS, Tyne and Wear (GB)

(73) Assignee: University of Newcastle Upon Tyne, Tyne and Wear (GB)

(21) Appl. No.: 16/472,670

(22) PCT Filed: Dec. 19, 2017
(86) PCT No.: PCT/GB2017/053807

§ 371 Date: Jun. 21, 2019

(30) Foreign Application Priority Data
Dec. 23, 2016 (GB) 1622116.0

(51) Int. Cl. A61K 31/497; A61P 1/16

(52) U.S. Cl. CPC A61K 31/497; A61P 1/16

(57) ABSTRACT

Aspects and embodiments of the present invention relate to the treatment of fatty liver diseases such as for example, Non-Alcoholic Fatty Liver Disease (NAFLD) and Non-Alcoholic Steatohepatitis (NASH). Particularly, but not exclusively certain embodiments relate to activators of glucokinase (GKAs) for use in the treatment of these fatty liver diseases. Also included in the present invention are inter alia pharmaceutical compositions comprising the GKAs, together with methods of treating such disorders as well as other subject matter.

### FIELD OF THE INVENTION

Aspects and embodiments of the present invention relate to the treatment of fatty liver diseases such as for example, Non-Alcoholic Fatty Liver Disease (NAFLD) and Non-Alcoholic Steatohepatitis (NASH). Particularly, but not exclusively certain embodiments relate to activators of glucokinase (GKAs) for use in the treatment of these fatty liver diseases. Also included in the present invention are inter alia pharmaceutical compositions comprising the GKAs, together with methods of treating such disorders as well as other subject matter.

### BACKGROUND TO THE INVENTION

Non-alcoholic fatty liver disease is characterized by macrovesicular steatosis of the liver occurring in individuals who typically consume little or no alcohol. The histological spectrum of NAFLD includes the presence of steatosis alone, fatty liver and inflammation and a more aggressive form called non-alcoholic steatohepatitis (NASH), characterized by the presence of steatosis, inflammation and varying degrees of fibrosis. NAFDL and NASH can be a precursor to cirrhosis of the liver which can lead to permanent liver damage, liver failure and liver cancer.

The risk of NAFLD may be increased in subjects who are obese or overweight, suffer from diabetes, have high blood pressure, have high cholesterol and/or smoke. However cases of NAFLD have been reported in subjects not having any of the listed factors. A lack of symptoms and uniform diagnosis criteria make diagnosis and prevalence of NAFLD difficult to determine. Currently it is believed that NASH may be present in 5% of the general population.

Currently there are no approved pharmacological treatments for NAFLD. Most commonly subjects suffering from NAFLD are advised to undertake lifestyle changes such as for example increasing exercise and adopting healthy eating habits. Alternatively treatment of the factors that increase the risk of NAFLD may be considered the best course of action. Despite these methods of treatment there is a clear need for additional new therapies.

Glucokinase activators (GKAs) are molecules that act to increase the activity of the protein glucokinase (GK). GK is one of the four members of the hexokinase family. It operates in the glucose/glucose 6-phosphate (G6P) cycle and phosphorylates glucose to form glucose-6-phosphate (G6P). Expression of GK is limited to the liver (parenchymal hepatocytes) and endocrine or neuroendocrine cells in the pancreatic islets, brain and the gastrointestinal tract, that have an integrated role in glucose sensing. In the liver, phosphorylation of glucose by GK promotes glycogen synthesis and glycolysis, whereas in the pancreatic β-cells, it is coupled to insulin release. GK has unique biochemical kinetics (it shows non-Michaelis-Menten kinetics) that accounts for its role as a glucose sensor. GK has a relatively low affinity for its substrate (glucose) and the GK protein is not directly inhibited by its end product G6P. Due to its role in glucose homeostasis GK has been a target protein for the treatment of Type 2 Diabetes (T2D).

GKAs act through a number of mechanisms to promote the activity of GK. They have been shown to bind to an allosteric site on GK. This binding maintains GK in an active “closed” conformation thereby increasing the affinity for glucose. This conformation reduces the binding of the liver GK regulator protein (GKRP) which binds to GK in the open inactive conformation and sequesters liver GK to the nucleus of the liver cell. By stabilizing the closed conformation GKAs favour the accumulation of liver GK into the cytoplasm where it operates. In addition, to increasing the affinity of GK for glucose some GKAs also increase the maximal activity of GK.

In animal models of T2D GKAs were shown to decrease blood glucose and reduce hyperglycaemia. Even so in Phase II clinical trials this efficacy of GKAs on lowering blood glucose dropped after an initial period of glucose reduction (in most cases between 4 to 10 weeks). This loss of effect meant that GKAs were not considered to be suitable for treating T2D and trials were discontinued. The cause of this drop in efficiency was not fully understood and it was suggested to be caused by an increase in lipid biosynthesis and an increase in triglycerides in both plasma and the liver. This argument was supported by evidence that attenuating mutations in GKRP which increased activity of GK also led hyperlipidaemia. Further studies of GKAs indicated that the amount of lipid elevation was dependant on the degree of GK activation. Even so the loss of efficiency in treating hyperglycaemia was still prevalent for those GKAs that did not raise overall lipid levels. This consistent loss of efficiency after a peak in activity seen for GKAs in the treatment of T2D and a lack of understanding of the cause of this has meant that several GKAs have been abandoned as a viable treatment for T2D.

It is an aim of the present invention to at least partly mitigate the problems associated with the prior art.

It is an aim of certain embodiments of the present invention to provide a treatment for liver diseases such as for example NAFLD.

### SUMMARY OF CERTAIN EMBODIMENTS OF THE INVENTION

According to a first aspect of the present invention there is provided a Glucokinase Activator (GKA) or a GK ligand which activates glucokinase (GK) or a pharmaceutically acceptable salt or derivative thereof for use in the treatment of a liver disease.

In certain embodiments, the GKA or GK ligand is for use in combination with at least one further therapeutic agent. Aptly the GKA and further therapeutic agent are for use in treating a subject suffering from a liver disease and type II diabetes and/or metabolic syndrome.

According to a second aspect of the present invention there is provided a composition, comprising a GKA or a GK ligand which activates glucokinase or a pharmaceutically acceptable salt or derivative thereof and one or more pharmaceutically acceptable carriers for use in the treatment of a liver disease.

In certain embodiments, the composition further comprises at least one further therapeutic agent.

Aptly the liver disease is a fatty liver disease. Aptly the fatty liver disease is selected from one or more of Non-alcoholic Fatty Liver Disease (NAFLD) and/or Non-alcoholic Steatohepatitis (NASH). Details of liver diseases which may be treated by the GKA are provided below.

Aptly the GKA is liver selective.

In certain embodiments the GKA is 3-{[5-(azetidin-1-carbonyl)pyrazin-2-yl]oxy}-5-{[(1S)-1-methyl-2-(methyloxy)ethyl]oxy}-N-(5-methylpyrazin-2-yl)benzamide or a pharmaceutically acceptable salt or derivative thereof.

According to a third aspect of the present invention there is provided a method of treating and/or preventing a liver disease comprising administering to a subject in need thereof a therapeutically effective amount of GKA or a pharmaceutically acceptable salt or derivative thereof or a pharmaceutical composition comprising the GKA and one or more pharmaceutically acceptable carriers.

Aptly the method, whereby the GKA is administered for a period of time greater than about four weeks.

Aptly the liver disease is a fatty liver disease.

Aptly the fatty liver disease is selected from one or more of Non-alcoholic Fatty Liver Disease (NAFLD) and/or Non-alcoholic Steatohepatitis (NASH).

Aptly the GKA is relatively liver selective.

In certain embodiments the GKA is 3-{[5-(azetidin-1-carbonyl)pyrazin-2-yl]oxy}-5-{[(1S)-1-methyl-2-(methyloxy)ethyl]oxy}-N-(5-methylpyrazin-2-yl)benzamide or a pharmaceutically acceptable salt or derivative thereof.

Aptly the method further comprises administering at least one further therapeutic agent. The at least one further therapeutic agent may be an agent for the treatment of diabetes e.g. Type II diabetes.

According to a fourth aspect of the present invention there is provided a method of increasing glucose-6-phosphate and/or fructose-2,6-bisphosphate concentration in a subject, comprising administering to the subject a GKA or a pharmaceutically acceptable salt or derivative thereof or a pharmaceutical composition comprising the GKA and one or more pharmaceutically acceptable carriers.

Aptly the method, whereby the GKA is administered for a period of time greater than four weeks.

Aptly the GKA is as described herein.

Aptly the method further comprises administering at least one further therapeutic agent. The at least one further therapeutic agent may be administered separately, simultaneously or sequentially.

According to a fifth aspect of the present invention there is provided a method of reducing GK activity in a subject, comprising administering to the subject a GKA or other GK ligand or a pharmaceutically acceptable salt or derivative thereof or a pharmaceutical composition comprising the GKA and one or more pharmaceutically acceptable carrier.

Aptly the method, whereby the GKA or other GK ligand is administered for a period of time greater than four weeks.

Aptly the GKA is as described herein.

In certain embodiments, the method further comprises administering at least one further therapeutic agent.

Aptly the GK is liver GK.

According to a sixth aspect of the present invention there is provided a method of treating and/or preventing steatosis in a subject in need thereof, comprising administering to the subject a GKA or a pharmaceutical composition comprising the GKA and one or more pharmaceutically acceptable carriers.

Aptly the method whereby the GKA is administered for a period of time greater than four weeks.

Aptly the GKA is as described herein.

Aptly the method further comprises administering at least one further therapeutic agent.

Aptly the steatosis is hepatic steatosis.

According to a seventh aspect of the present invention there is provided a method of screening for a therapeutic agent for use in the treatment of a liver diseases comprising the steps of, contacting a candidate agent with at least one hepatocyte preparation and measuring the expression of liver Gck mRNA.

### BRIEF DESCRIPTION OF DRAWINGS

Certain embodiments of the present invention will now be described hereinafter, by way of example only, with reference to the accompanying drawings in which:

FIG. 1 illustrates the effects of a GKA on glucose metabolism and gene expression in hepatocytes of mammals that express glucokinase as the predominant hexokinase such as man and most preclinical models such as mice and rats;

FIG. 2 shows graphs that illustrate the effects of the GKA RO 28-1675 on glucose phosphorylation (2A), glucose-6-phosphate concentration (2B) of hepatocytes incubated for 2 hours in MEM containing 10 nM insulin and basal glucose (5 mM glucose or 5G), medium glucose (15 mM glucose or 15G) or high glucose (25 mM or 25G) and +/−10 μM GKA. At all glucose concentrations the GKA: increased phosphorylation more than glucose alone; and increased the concentration of G6P more than glucose alone. The error bars show ±standard error of mean (SEM), n=4-6, *P<0.05 effect of GKA;

FIG. 3 shows graphs that illustrate the effects of the GKA with 15 mM glucose (15 G) in comparison to the effects of 5 mM glucose (5 G) and 15 mM glucose (15 G) alone on fructose 2,6-bisphosphate concentration (3A) and relative expression of G6pc mRNA (3B), Pklr mRNA (3C), Gck mRNA (3D). Hepatocytes were incubated for 4 hours without (open bars) or with (filled bars) 10 nM insulin. The GKA increases F-2,6-P2 more than 5 mM glucose and 15 mM glucose. The GKA also increased relative expression of Pklr mRNA (3C) more than 5 mM glucose and 15 mM glucose. The GKA decreased relative expression of Gck mRNA (3D) in comparison to 5 mM glucose and 15 mM glucose. Means±SEM, n=3-6, #P<0.05 relative to 15 mM glucose;

FIG. 4 shows graphs that illustrate the effects of the GKA on relative expression of Gck mRNA (4A), Pklr mRNA (4B) and G6pc mRNA (4C) at 5 mM, 15 mM and 25 mM glucose. Hepatocytes were incubated for 4 hours with 10 nM insulin and 5,15 or 25 mM glucose (G) −/+10 μM GKA. The GKA reduced the relative expression of Gck mRNA at all glucose concentrations and increased relative expression of Pklr mRNA and G6pc mRNA at all glucose concentrations. Means±SEM, n=3, *P<0.05 effect of GKA;

FIG. 5 shows graphs that illustrate the correlation between glucose 6-phosphate concentration and relative expression of Gck mRNA, Pklr mRNA and G6pc mRNA. The data shown for relative expression in FIG. 4 is plotted against the respective concentration of G6P in each experiment. Relative expression of Gck mRNA has a negative correlation with concentration of G6P while relative expression of Pklr and G6pc mRNA show a positive correlation with G6P concentration.

FIG. 6 shows a diagram that illustrates the effects of GK phosphorylation of glucose and 2-deoxyglucose (DG) (6A). The effects of the H6P transport inhibitor S4048 which blocks the hydrolysis of the hexose 6-phosphate (H6P) products (G6P and 2-deoxyglucose 6-phosphate (2DG6P) is also illustrated (6A). FIG. 6 also shows graphs that illustrate the effects of DG on H6P concentration (6B) and the effect H6P concentration has on G6pc mRNA relative expression (6C) and Gck mRNA relative expression (6D) and how the levels of Gck mRNA relative expression correlates with H6P levels (6E). H6P concentration increases with increasing 2DG and more so when the inhibitor S4048 is present (6B). The increase in H6P concentration does not affect G6pc mRNA relative expression (6C) but does lead to a decrease in Gck mRNA expression and more so when the inhibitor S4048 is present (6D). This decrease in Gck expression correlates with the concentration of H6P (6E). *P<0.05 relative to 5 G; #P<0.05 relative to 25G.

### DETAILED DESCRIPTION OF CERTAIN EMBODIMENTS

Further details of embodiments of the invention are provided below.

Most general molecular biology, microbiology recombinant DNA technology and immunological techniques can be found in Sambrook et al, Molecular Cloning, A Laboratory Manual (2001) Cold Harbor-Laboratory Press, Cold Spring Harbor, N.Y. or Ausubel et al., Current protocols in molecular biology (1990) John Wiley and Sons, N.Y. Unless defined otherwise, all technical and scientific terms used herein have the same meaning as commonly understood by one of ordinary skill in the art to which this disclosure belongs. For example, the Concise Dictionary of Biomedicine and Molecular Biology, Juo, Pei-Show, 2^{nd} ed., 2002, CRC Press; The Dictionary of Cell and Molecular Biology, 3^{rd} ed., Academic Press; and the Oxford University Press, provide a person skilled in the art with a general dictionary of many of the terms used in this disclosure.

According to a first aspect of the present invention there is provided a Glucokinase Activator (GKA) or a GK ligand or a pharmaceutically acceptable salt or derivative thereof for use in the treatment of a liver disease. In certain embodiments, the liver disease may be non-alcoholic fatty liver disease (NAFLD). Alternatively, in certain embodiments, the liver disease may be non-alcoholic steatohepatitis (NASH).

As described herein the term “liver disease” may refer to a fatty liver disease. Fatty liver diseases are typically conditions wherein large vacuoles of triglyceride fat accumulate in liver cells via the process of steatosis (i.e., abnormal retention of lipids within a cell). Accumulation of fat may also be accompanied by a progressive inflammation of the liver (hepatitis), called steatohepatitis. By considering the contribution of alcohol, fatty liver disease may be termed alcoholic steatosis or non-alcoholic fatty liver disease (NAFLD).

NAFLD refers to a condition where there is an accumulation of excess fat and/or lipids in the liver of people whose alcohol ingestion is not enough to cause liver injury, except for cases of known etiology, such as viral hepatitis and autoimmune hepatitis.

NAFLD is more likely to present itself in individuals who present one or more risk factors such as for example, elevated fasting plasma glucose (FPG) with or without intolerance to post-prandial glucose, being overweight or obese, diabetes or pre-diabetes, high blood lipids such as cholesterol and triglycerides (TGs) and low high-density lipoprotein cholesterol (HDL-C) levels and/or high blood pressure; but not all patients present all or any of these risk factors. The majority of individuals with NAFLD have no symptoms and appear asymptomatic upon physical examination (although the liver may be slightly enlarged). Children may exhibit symptoms such as abdominal pain and fatigue, and may show patchy dark skin discoloration (acanthosis nigricans). The diagnosis of NAFLD is usually first suspected in an overweight or obese person who is found to have mild elevations in their liver blood tests during routine testing, though NAFLD can be present with normal liver blood tests, or incidentally detected on imaging investigations such as abdominal ultrasound or CT scan. It is usually confirmed by imaging studies, such as a liver ultrasound or magnetic resonance imaging (MRI).

In some cases NAFLD may progress to a more serious condition called non-alcoholic steatohepatitis (NASH). In NASH, fat and/or lipid accumulation in the liver is associated with inflammation and different degrees of scarring. NASH is a potentially serious condition that carries a substantial risk of progression to end-stage liver disease, cirrhosis and hepatocellular carcinoma. Some patients who develop cirrhosis are at risk of liver failure and may eventually require a liver transplant.

NAFLD and NASH are usually associated with metabolic diseases such as for example one or more of mixed dyslipidemia, obesity, hypercholesterolemia, hyperlipidemia, hypertriglyceridemia as well as other disorders that are associated with abnormal levels of lipoproteins, lipids, carbohydrates and insulin such as metabolic syndrome X, diabetes, impaired glucose tolerance, atherosclerosis, coronary artery disease, cardiovascular disease and polycystic ovary syndrome (PCOS).

NAFLD may be differentiated from NASH by the NAFLD Activity Score (NAS), the sum of the histopathology scores of a liver biopsy for steatosis (0 to 3), lobular inflammation (0 to 2), and hepatocellular ballooning (0 to 2). A NAS of <3 corresponds to NAFLD, 3-4 corresponds to borderline NASH, and 5 corresponds to NASH. The biopsy may also scored for fibrosis (0 to 4).

As used herein the term “metabolic syndrome X” also known as “metabolic syndrome” and “dysmetabolic syndrome” refers to a condition identified by the presence of three or more of the components:
Central obesity as measured by waist circumference:
   Men: Greater than 40 inches Women: Greater than 35 inches.
Blood triglycerides greater than or equal to 150 mg/dL.
Blood HDL cholesterol:
   Men: Less than 40 mg/dL Women: Less than 50 mg/dL.
Blood pressure greater than or equal to 130/85 mmHg.
Fasting blood glucose greater than or equal to 110 mg/dL.

In certain embodiments the GKAs are for use in the treatment of a liver disease in a subject in need of treatment who suffers from diabetes. As used herein the term “diabetes” refers to metabolic defects in the production and utilization of carbohydrates, particularly glucose, which result in the failure of glucose homeostasis such as for example Type I diabetes, or insulin-dependent diabetes mellitus (IDDM) which results from the absolute deficiency of insulin and Type II diabetes, or non-insulin dependent diabetes mellitus (NIDDM), which often occurs with normal or elevated levels of insulin and appears to be the result of the inability of cells and tissues to respond appropriately to insulin. Other forms of diabetes will be known by those skilled in the art.

In certain embodiments the GKAs are for use in the treatment of a liver disease in a subject in need of treatment that is pre-diabetic but does not yet suffer from diabetes. Subjects with glucose levels between normal and diabetic may have impaired glucose tolerance. Depending on the level of glucose intolerance this condition may be called pre-diabetes or insulin resistance syndrome. Subjects with pre-diabetes do not have diabetes, but rather have blood glucose levels that are higher than normal but not yet high enough to be diagnosed as diabetes.

It is considered, without being bound by theory, glucokinase (GK) is associated with fatty liver disease. It has been observed that increased expression of the liver GK encoding gene (Gck) and liver GK activity may be associated with triglyceride, fatty acid and lipid levels in the liver and hepatic steatosis. GK activity and Gck expression have also been suggested to be positively correlated with the expression of lipogenic genes such as for example, fatty acid synthase, acetyl-coenzyme A carboxylase-a and acetyl-coenzyme A carboxylase-β. Increase in lipogenesis may lead to an increase in hepatic steatosis and therefore lead to NAFLD and NASH.

GKAs are considered to decrease the expression of the liver GK gene (Gck). The reduction of expression of liver Gck may lead to a lower concentration of liver GK and a decrease in the activity of liver GK. Therefore, certain embodiments of the present invention are based on the inventors' finding that GKAs may be helpful in reducing steatosis by reducing the expression of lipogenic genes and lipogenesis and therefore may act on liver GK and Gck expression thus having utility in the treatment of liver diseases, such as NAFLD and/or NASH.

Thus GKAs may therefore have utility in the treatment of disorders associated with steatosis.

GK, also known as hexokinase IV or D is an enzyme that, among other things, facilitates phosphorylation of glucose to glucose-6-phosphate in the process of glycolysis. In vertebrates, GK-mediated phosphorylation generally occurs in the parenchymal hepatocytes in the liver, where it mediates hepatic glucose clearance and in endocrine and neuroendocrine cells in the pancreatic islets, gut, and brain, where it functions as a glucose sensor, triggering shifts in metabolism or cell function in response to rising and/or falling levels of blood-glucose. GK functions in a dual role, with distinct functions in the pancreatic islets and liver; (a) as a molecular glucose sensor in the insulin-producing pancreatic β-cells, and (b) as the high-capacity enzymatic step initiating the storage of glucose in the form of glycogen in the liver and uptake of glucose during hyperglycemia. Therefore, GK plays a central role in glucose homeostasis, through the phosphorylation of glucose in the liver, and the modulation of insulin secretion in the pancreas.

GK, has two main distinctive characteristics: (1) its expression, which is limited to tissues that require glucose-sensing (mainly liver and pancreatic β-cells), and (2) its sigmoidal saturation curve, which has a value of S_{0.5} for glucose that is much higher (8-12 mM) than that of the other members of the hexokinase family. Due to these kinetic characteristics, changes in blood glucose levels are paralleled by changes in glucose metabolism in liver, which in turn regulates the balance between hepatic glucose output (HGO) and glucose consumption. Tissue-specific differences between GK regulation in liver and pancreatic β-cells have been observed. In liver, GK gene (Gck) transcription is stimulated by insulin and inhibited by glucagon. In the liver the activity of GK is also modulated by a glucokinase regulatory protein (GKRP), which binds and inhibits GK competitively with respect to glucose. Under basal glucose conditions (˜5 mM) hepatic GK is bound largely to GKRP and it is located in the nucleus of the hepatocytes. However, after exposure to either high glucose (10-30 mM) or fructose (50 μM to 2 mM), hepatic GK is released from the GKRP and exits the nucleus in an active “closed” state. In the pancreatic β-cells, GK gene expression is thought to be largely constitutive, although glucose modulates pancreatic β-cells GK content, probably by directly affecting the half-life of the enzyme since GKRPs have not been shown on pancreatic β-cells.

Sequences for glucokinase are known in the art and may be found in the following database:
1. Human Liver Glucokinase

UNIPROT: P35557.

Isoforms 2 and 3 are found in the liver.

Glucokinase activators (GKAs) are compounds or agents that activate GK activity in a subject, such as a human, in direct or indirect response to the presence of the compound or agent, or a metabolite thereof in the subject. As GK is closely associated with glucose homeostasis GKAs have been extensively studied for use in the treatment glucose homeostasis disorders such as hyperglycaemia and type II diabetes (T2D). The GKA may be a small molecule agent or other type of GK ligand which activates GK.

In pancreatic β-cells GKAs have been shown to increase insulin release and therefore increase glucose uptake by the liver and therefore decreasing blood glucose levels. In liver cells GKAs promote phosphorylation of glucose and thereby hepatic glucose clearance. Certain GKAs have been proposed to increase GK activity through a number of mechanism: in both liver and pancreatic β-cells by: (i) binding to an allosteric site on GK and maintaining GK in its active “closed” state; (ii) increasing GK affinity for glucose and in liver cells specifically GKAs have been proposed to increase the release of GK from GKRP and therefore GK translocation to the cytoplasm.

Thus, in one aspect of the present invention there is provided a method of treating and/or preventing a liver disease comprising administering to a subject in need thereof a therapeutically effective amount of GKA or a pharmaceutically acceptable salt or derivative thereof or a pharmaceutical composition comprising the GKA and one or more pharmaceutically acceptable carriers. In certain embodiments, the liver disease is a fatty liver disease. Aptly the fatty liver disease is selected from one or more of NAFDL and/or NASH.

In another aspect of the present invention there is provided a method of treating and/or preventing steatosis in a subject in need thereof, comprising administering to the subject a GKA or a pharmaceutical composition comprising the GKA and one or more pharmaceutically acceptable carriers. Aptly the steatosis is hepatic steatosis.

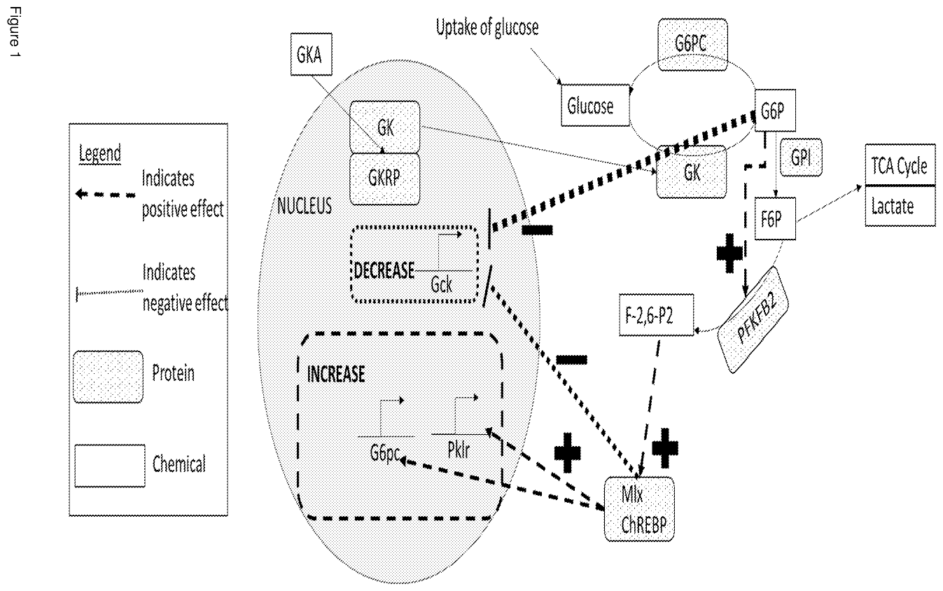

A mechanism for the repression of Gck is illustrated in the diagram shown in FIG. 1. It is considered that the GKA promotes the dissociation of glucokinase (GK) from glucokinase regulating protein (GKRP) which then leads to GK being transported from the nucleus to the cytoplasm. GK then mediates the conversion of glucose to glucose-6-phosphate (G6P). The raised level of glucose-6-phosphate, the product of the glucokinase reaction, causes repression of the liver Gck gene.

G6P is converted by phosphoglucose isomerase (GPI) to fructose-6-phosphate (F6P). G6P positively stimulates phosphofructokinase 2/fructose-2,6-bisphosphatase (PFKFB2) which increases the conversion of fructose-6-phosphate to Fructose-2,6-bisphosphate (F-2,6-P₂).

It is considered that F-2,6-P₂ promotes the binding of Max like protein linked transcription factors e.g. the carbohydrate responsive element binding protein (Mix ChREBP) to its target genes. Mix ChREBP promotes the expression of glucose-6-phosphotase (G6PC) gene G6pc (which catalyses the reconversion of the product of the glucokinase reaction (glucose 6-phosphate) back to glucose). Mix ChREBP may also promote the expression of the pyruvate kinase gene (Pklr), which may help deplete phosphate ester intermediates.

In one aspect of the present invention there is provided a method of reducing GK activity in a subject, comprising administering to the subject a GKA. Aptly, the GKA is administered for a period of time sufficient to reduce mRNA expression of GK in the subject. Aptly, the method is for reducing GK activity in the subject's liver.

Thus, in another aspect of the invention there is provided a method of increasing glucose 6-phosphate and/or fructose-2,6-bisphosphate in a subject, comprising administering to the subject a GKA or a pharmaceutical composition comprising the GKA and one or more pharmaceutically acceptable carriers to the subject.

One skilled in the art will appreciate that there are a range of molecules or agents, which would promote the activity of glucokinase and thus may have utility in certain embodiments of the present invention. However, a non-exhaustive list of specific examples may include an agent independently selected from:

3-{[5-(azetidin-1-carbonyl)pyrazin-2-yl]oxy}-5-{[(1S)-1-methyl-2-(methyloxy)ethyl]oxy}-N-(5-methylpyrazin-2-yl)benzamide;

6-[(3-isobutoxy-5-isopropoxybenzoyl)amino]nicotinic acid;

5-({3-isopropoxy-5-[2-(3-thienyl)ethoxy]benzoyl}amino)-1,3,4-thiadiazole-2- carboxylic acid;

N-Benzothiazol-2-yl-2(R)-(3-chloro-4-methanesulfonyl-phenyl)-3-cyclopentyl-propionamide;

N-(1H-benzimidazol-2-yl)-2-(3-chloro-4-methanesulfonyl-phenyl)-3-cyclopenty-1-propionamide;

2-(3-chloro-4-methanesulfonyl-phenyl)-3-cyclopentyl-N-quinolin-2-yl-propionamide;

N-(5-bromo-pyridin-2-yl)-3-cyclopentyl-2-[3-chloro-4-(5-methyl-tetrazol-1-yl)-phenyl]-propionamide;

N-(5-bromo-pyridin-2-yl)-3-cyclopentyl-2-[4-(5-methyl-tetrazol-1-yl)-3-trifluoromethyl-phenyl]-propionamide;

3-Cyclopentyl-2-[4-(5-methyl-tetrazol-1-yl)-3-trifluoromethyl-phenyl]-thiazol-2-yl-propionamide;

(E)-2-[3-chloro-4-(5-methyl-tetrazol-1-yl)-phenyl]-3-cyclohexyl-N-thiazol-2-yl- acrylamide;

(E)-2-[3-chloro-4-(5-trifluoromethyl-tetrazol-1-yl)-phenyl]-3-cyclohexyl-N-thiazol-2-yl-acrylamide;

(E)-2-[3-chloro-4-(5-methyl-tetrazol-1-yl)-phenyl]-3-cycloheptyl-N-thiazol-2-yl-acrylamide;

(E)-N-(5-bromo-thiazol-2-yl)-2-[3-chloro-4-(5-methyl-tetrazol-1-yl)-phenyl]-3-cycloheptyl-acrylamide;

(E)-2-[3-chloro-4-(5-methyl-tetrazol-1-yl)-phenyl]-3-cyclopentyl-N-thiazol-2-yl- acrylamide;

(E)-3-cyclopentyl-2-[3-fluoro-4-(5-methyl-tetrazol-1-yl)-phenyl]-N-thiazol-2-yl- acrylamide;

3-cyclopentyl-2-[3-fluoro-4-(5-methyl-tetrazol-1-yl)-phenyl]-N-thiazol-2-yl- propionamide;

2-[3-chloro-4-(5-methyl-tetrazol-1-yl)-phenyl]-3-cyclopentyl-N-thiazol-2-yl- propionamide;

3-cyclopentyl-2-[4-(5-methyl-tetrazol-1-yl)-3-trifluoromethyl-phenyl]-thiazol-2-yl-propionamide;

(E)-3-cyclopentyl-2-[4-(5-methyl-tetrazol-1-yl)-3-trifluoromethyl-phenyl)-]-N-thiazol-2-yl-acrylamide;

(E)-2-[3-chloro-4-(5-methyl-tetrazol-1-yl)-phenyl]-3-cyclopentyl-N-thiazol-2-yl-acrylamide;

(E)-3-cyclopentyl-2-[3-fluoro-4-(5-methyl-tetrazol-1-yl)-phenyl]-N-thiazol-2-yl-acrylamide;

3-Cyclopentyl-2-(4-methanesulfonyl-phenyl)-N-thiazol-2-yl-propionamide;

3-Cyclopentyl-N-thiazol-2-yl-2-(4-trifluoromethoxy-phenyl)-propionamide;

3-Cyclopentyl-N-thiazol-2-yl-2-(4-trifluoromethanesulfonyl-phenyl)-propionamide;

3-Cyclopentyl-2(R)-(3,4-dichloro-phenyl)-N-pyridin-2-yl-propionamide;

6-[3-Cyclopentyl-2(R)-(3,4-dichloro-phenyl)-propionylamino]-nicotinic acid methyl ester;

N-(5-Chloro-pyridin-2-yl)-3-cyclopentyl-2(R)-(3,4-dichloro-phenyl)-propionamide;

3-Cyclopentyl-N-pyridin-2-yl-2-(4-trifluoromethanesulfonyl-phenyl)-propionamide;

3-Cyclopentyl-N-(5-methyl-pyridin-2-yl)-2-(4-trifluoromethanesulfonyl-phenyl)- propionamide;

3-Cyclopentyl-2(R)-(3,4-dichloro-phenyl)-N-(5-hydroxymethyl-pyridin-2-yl) propionamide;

6-[3-Cyclopentyl-2-(4-trifluoromethanesulfonyl-phenyl)-propionylamino]-nicotinic acid methyl ester;

3-Cyclopentyl-2-(3-fluoro-4-trifluoromethyl-phenyl)-N-pyridin-2-yl-propionamide;

3-Cyclopentyl-2-(4-methanesulfonyl-3-nitrophenyl)-N-pyridin-2-yl-propionamide;

2-(3-Bromo-4-methanesulfonyl-phenyl)-3-cyclopentyl-N-pyridin-2-yl-propionamide;

2-(3-Cyano-4-methanesulfonyl-phenyl)-3-cyclopentyl-N-pyridin-2-yl-propionamide;

2-(4-Chloro-3-nitro-phenyl)-3-cyclopentyl-N-pyridin-2-yl-propionamide;

2-(3-Chloro-4-methanesulfonyl-phenyl)-3-cyclopentyl-N-pyridin-2-yl-propionamide;

N-(5-Bromo-pyridin-2-yl)-2-(3-chloro-4-methanesulfonyl-phenyl)-3-cyclopentyl-propionamide;

2-[3-Chloro-4-methanesulfonyl-phenyl]-3-cyclopentyl-N-thiazol-2-yl-propionamide;

(2R)-3-Cyclopentyl-2-(4-methanesulfonylphenyl)-N-thiazol-2-yl-propionamide;

2-(3-Bromo-4-methanesulfonyl-phenyl)-3-cyclopentyl-N-thiazol-2-yl-propionamide;

2-(3-Cyano-4-methanesulfonyl-phenyl)-3-cyclopentyl-N-thiazol-2-yl-propionamide;

3-Cyclopentyl-2-(4-ethanesulfonyl-phenyl)-N-thiazol-2-yl-propionamide;

3-Cyclopentyl-2-(4-methanesulfonyl-3-trifluoromethyl-phenyl)-N-thiazol-2-yl-propionamide;

[7-Cyclopentylamino-2-((R)-4-hydroxymethyl-4,5-dihydro-thiazol-2-yl)-1H-indol-5-yl]-methanol;

3-Cyclopentyl-2-(4-ethynyl-phenyl)-N-thiazol-2-yl-propionamide;

3-Cyclopentyl-2-[4-(1-hydroxy-cyclohexylethynyl)-phenyl]-thiazol-2-yl- propionamide;

3-Cyclopentyl-2-[4-(3-methoxy-prop-1-ynyl)-phenyl]-N-thiazol-2-yl-propionamide;

3-Cyclopentyl-2-[4-(3-hydroxy-3-methyl-pent-1-ynyl)-phenyl]-N-thiazol-2-yl- propionamide;

3-Cyclopentyl-2-[4-(4-hydroxy-pent-1-ynyl)-phenyl]-N-thiazol-2-yl-propionamide;

3-Cyclopentyl-2-[4-(3-hydroxy-prop-1-ynyl)-phenyl]-N-thiazol-2-yl-propionamide;

3-Cyclopentyl-2-14-(3-dimethylamino-prop-1-ynyl)-phenyl)-N-thiazol-2-yl- propionamide;

3-Cyclopentyl-2-[4-(3-morpholin-4-yl-prop-1-ynyl)-phenyl]-N-thiazol-2-yl- propionamide;

2-Biphenyl-4-yl-3-cyclopentyl-N-thiazol-2-yl-propionamide;

2-(2-Biphenyl-4-yl-3-cyclopentyl-propionylamino)-thiazole-4-carboxylic acid methyl ester;

2-Biphenyl-4-yl-3-cyclopentyl-N-(4-hydroxymethyl-thiazol-2-yl)-propionamide;

(2R)-2-Biphenyl-4-yl-3-cyclopentyl-N-thiazol-2-yl-propionamide;

3-Cyclopentyl-2-(4-naphthalen-1-yl-phenyl)-N-thiazol-2-yl-propionamide;

3-Cyclopentyl-N-[4-(2-hydroxyethyl)-thiazol-2-yl]-2-(4-naphthalen-1-yl-phenyl)-propionamide;

3-Cyclopentyl-N-thiazol-2-yl-2-(4-thiophen-2-yl-phenyl)-propionamide;

3-Cyclopentyl-2-(4-pyridin-3-yl-phenyl)-N-thiazol-2-yl-propionamide; and

3-Cyclopentyl-2-[4-(IH-indol-5-yl)-phenyl]-N-thiazol-2-yl-propionamide.

Other examples of GKAs and methods of producing the same can be found in WO2010092386, WO2007/061923A2, WO2010103438A1 and WO2011149945 and the compounds described therein which are hereby expressly incorporated herein by reference.

In certain embodiments the GKA is 3-[{5-(azetidin-1-carbonyl)pyrazin-2-yl]oxy}-5-{[(1S)-1-methyl-2-(methyloxy)ethyl]oxy}-N-(5-methylpyrazin-2-yl)benzamide or a pharmaceutically acceptable salt or derivative thereof. 3-[{5-(azetidin-1-carbonyl)pyrazin-2-yl]oxy}-5-{[(1S)-1-methyl-2-(methyloxy)ethyl]oxy}-N-(5-methylpyrazin-2-yl)benzamide may also be known in the art as AZD1656.

In certain embodiments, the GKA may be a liver selective GKA. A liver-selective GKA may directly or indirectly increases glucose utilization in the liver at doses that do not induce a substantial increase in insulin secretion by the pancreas in response to glucose (e.g., less than a 25% increase, or less than a 15% increase, or less than a 10% increase, or less than a 5% increase, or less than a 3% increase in insulin secretion by the pancreas in response to glucose).

One skilled in the art will appreciate that there are a range of molecules or agents, which would be suitable liver selective GKAs. However, a non-exhaustive list of examples may include an agent independently selected from:

2-[3-cyclohexyl-3-(trans-4-propoxy-cyclohexyl)-ureido]-thiazole-5-ylsulfanyl}-acetic acid;

2-{3-[2-(2, 3-dimethoxyphenoxy)-5-fluorophenyl] ureido}thiazole-4-carboxylic acid ethyl ester;

(2-{3-[2-(2, 3-dimethoxyphenoxy)-5-fluorophenyl] ureido}thiazol-4-yl) acetic acid ethyl ester;

(2-{3-[5-fluoro-2-(2-fluoro-6-methoxyphenoxy) phenyl] ureido}thiazol-4-yl) acetic acid;

2-{3-[2-(2, 3-dimethoxyphenoxy)-5-fluorophenyl] ureido}thiazole-4-carboxylic acid;

2-(2-{3-[5-fluoro-2-(2-fluoro-6-methoxyphenoxy) phenyl] ureido}thiazol-4-yl)-N-(2-morpholin-4-ylethyl) acetamide;

[2-(2-{3-[5-fluoro-2-(2-fluoro-6-methoxyphenoxy) phenyl] ureido}thiazol-4-yl) acetylamino] acetic acid;

(S)-6-(3-Cyclopentyl-2-(4-(trifluoromethyl)-1H-imidazol-1-yl)propanamido)nicotinic Acid;

{2-[3-(2-cyclopentanecarbonyl-4-methyl-phenyl)-ureido]-thiazol-4-yl}acetic acid; and

{2-[3-(4-methyl-2-[2-methylpropoxy] phenyl)-ureido]-thiazol-4-yl}-acetic acid.

Other examples of liver selective GKAs and methods of producing the same can be found in WO2010029461, WO2009023718 and WO2004002481 and the compounds described therein which are hereby expressly incorporated herein by reference.

In certain embodiments, a GK ligand may be used in the methods and treatment described herein. In certain embodiments, the GK ligand may be for example an antibody or fragment thereof. In certain embodiments, the GK ligand may be a nucleic acid molecule for example a nucleic acid aptamer. In certain embodiments, the GK ligand may be a peptide aptamer.

Even though GKAs have been shown to be initially effective at reducing blood glucose levels in diabetes models a loss of efficiency in blood glucose reduction was noted for GKAs in phase II trials. This loss of efficiency was noted after a period of days for some GKAs and up to 12 weeks for others. Certain GKAs were also shown to increase levels of lipids and triglycerides in plasma and liver and increase hepatic steatosis. This was believed to be the cause for the loss efficiency in reducing blood glucose. Due to the above-mentioned effects of GKAs and the loss of efficiency on blood glucose lowering the use of some certain GKAs in the treatment of diabetes was abandoned. It was observed though that even for those GKAs that did not raise lipids, and triglycerides levels the loss of efficiency on blood glucose lowering still occurred.

Without being bound by theory GKAs have been observed to increase the levels of G6P which directly and/or indirectly represses expression of Gck mRNA and directly and/or indirectly induces expression of the Glucose-6-phosphatase (G6PC) gene G6pc. G6PC converts G6P to glucose, therefore opposing the glucokinase reaction by removal of its product.

It is considered, without being bound by theory, half-life of the liver GK protein is longer than the half-life of liver Gck mRNA and so loss of GKA efficiency in reducing blood glucose may be delayed until liver GK is degraded as the level of liver GK may not decrease initially after repression of the liver Gck mRNA. This delay in loss of efficiency may be further extended by GKAs that cause the release of insulin from β-pancreatic cells as insulin induces the expression of liver Gck mRNA therefore counteracting the repressive effects of the raised G6P on the Gck gene and effects of raised activity of G6PC.

It is considered without being bound by theory that the counter intuitive repression of Gck and simultaneous induction of G6pc in response to high blood and/or liver glucose (hyperglycaemia) may be rationalised as a mechanism for preserving metabolite homeostasis at the expense of worsening hyperglycaemia.

Thus, in certain embodiments the GKA is administered for a period of time greater than the period of time in which reduction of blood glucose is induced by the GKA.

In certain embodiments, the GKA or a pharmaceutical composition comprising the GKA and one or more pharmaceutically acceptable carriers is administered for at least 1, 2, 3, 4, 5 or 6 or more days.

In certain embodiments, the GKA or a pharmaceutical composition comprising the GKA and one or more pharmaceutically acceptable carriers is administered for at least 1, 2, 3, 4, 5, 6, 7, 8, 9, 10, 11, 12, 13, 14, 15, 16, 17, 18, 19 or 20 weeks or more.

In certain embodiments, the GKA or a pharmaceutical composition comprising the GKA and one or more pharmaceutically acceptable carriers is administered for a period of time substantially equal to or greater than 4 weeks.

In certain embodiments, the GKA or a pharmaceutical composition comprising the GKA and one or more pharmaceutically acceptable carriers is administered in combination with at least one further therapeutic agent.

The term “further therapeutic agent” as used herein includes agents (other than the GKAs of certain embodiments of the present invention) such as at least one or more antidiabetic agents, one or more anti-obesity agents, anti-hypertensive agents, anti-platelet agents, anti-atherosclerotic agents and/or one or more lipid-lowering agents (including anti-atherosclerosis agents).

In subjects suffering from glucose homeostasis linked diseases such as for example diabetes (e.g. T2D), metabolic syndrome, hyperglycaemia or pre-diabetes and NAFLD and/or NASH or at risk of hepatic steatosis, NAFLD and/or NASH, the GKA or a pharmaceutical composition comprising the GKA may be administered in combination with one or more antidiabetic agents such as for example a sodium-glucose co-transporter 2 inhibitor (SGLT2 inhibitor). In certain embodiments, the GKA is for administration with other suitable anti-diabetic agents as will be known by those skilled in the art.

Thus in certain embodiments the method of treating and/or preventing a liver disease comprises administering to a subject in need thereof a therapeutically effective amount of GKA or a pharmaceutically acceptable salt or derivative thereof or a pharmaceutical composition comprising the GKA and one or more pharmaceutically acceptable carriers further comprises administering at least one antidiabetic agent.

In certain embodiments the method of treating and/or preventing steatosis in a subject in need thereof, comprising administering to the subject a GKA or a pharmaceutical composition comprising the GKA and one or more pharmaceutically acceptable carriers further comprises administering an anti-diabetic agent.

In certain embodiments the method of increasing glucose 6-phosphate and/or fructose-2,6-bisphosphate in a subject, comprising administering to the subject a GKA or a pharmaceutical composition comprising the GKA and one or more pharmaceutically acceptable carriers further comprises administering an anti-diabetic agent.

As used herein, the term “anti-diabetic agent” refers to an agent (other than the GKAs of certain embodiments of the present invention) capable of preventing or treating diabetes, for example type 2 diabetes and/or one or more diabetes related conditions or diseases such as for example hyperglycaemia, glucose intolerance, pre-diabetes, metabolic syndrome and/or insulin resistance. These agents are also useful for the treatment or prevention of associated symptoms. Antidiabetic agents may include one or more compounds belonging to one of the following classes: Gliflozins (SGLT2 inhibitors), canagliflozin, dapagliflozin, empagliflozin, DPP-4 inhibitors (gliptins), such as sitagliptin, vildagliptin, saxagliptin, alogliptin, linagliptin, gemigliptin, teneligliptin, trelagliptin, omarigliptin, biguanidines, metformin, thiazolidinediones (glitazones), such as pioglitazone, rosiglitazone, alpha-glucosidase inhibitors, such as acarbose, voglibose, miglitol.

Aptly the antidiabetic agent is an antidiabetic agent that does not stimulate the pancreas. Thus, in certain embodiments, the antidiabetic agent is not a sulphonulureas.

The term “in combination with” as used herein refers to in the course of treating and/or preventing a liver diseases as described herein and any other disease or risk factor for a liver disease, such as diabetes, obesity, insulin resistance, metabolic syndrome, hyperlipidaemia, hypercholesterolemia in the same subject and is intended to include simultaneous administration of the GKA and at least one further therapeutic agent, administration of the GKA first, followed by the further therapeutic agent and administration of the further therapeutic agent first, followed by the GKA. The further therapeutic agent can be administered by the same or one or more different routes than the GKA. The GKA and further therapeutic agent may be administered at an appropriate interval (e.g., an interval selected such that the GKAs of certain embodiments of the present invention and the further therapeutic agent are allowed to perform their intended function, e.g., the GKA and the further therapeutic agent are allowed to interact synergistically).

In certain embodiments the GKA and the antidiabetic agent is administered sequentially. Thus in certain embodiments, the anti-diabetic agent is administered following the GKA, wherein the period of time between initiation of treatment with the GKA and the initiation of treatment with the anti-diabetic agent is based on the reduction of blood glucose induced by the GKA (i.e. after the GKA loses efficiency in reducing blood glucose).

In certain embodiments, treatment with the anti-diabetic agent is initiated at least 1, 2, 3, 4, 5 or 6 days after initiation of treatment with the GKA. Aptly the period of time is substantially equal to or greater than 1, 2, 3, 4, 5, 6, 7, 8, 9, 10, 11, 12, 13, 14, 15, 16, 17, 18, 19 or 20 weeks.

In certain embodiments the period of time is 4 weeks.

According to a further aspect of the present invention there is provided a method of screening for a therapeutic agent for use in the treatment of a liver diseases comprising contacting a candidate agent with at least primary hepatocytes and measuring the expression or relative expression of liver Gck mRNA or liver glucokinase activity. Aptly the liver disease is a fatty liver disease. Aptly the liver disease is NAFLD and/or NASH. In certain embodiments the therapeutic agent is a GKA. In certain embodiments, the candidate agent reduces the expression of liver Gck mRNA.

Certain embodiments of GKAs of the present invention repress the expression of liver Gck mRNA.

Thus in certain embodiments of the present invention, there is provided a method of identifying agents which may be used to treat liver diseases as described herein. The method may be for example protein-based or cell-based. For example, a method may comprise exposing at least one liver cell to a candidate compound and detecting the level of liver Gck mRNA expression.

In some embodiments, the method comprises designing potential candidate compounds. The person skilled in the art will appreciate that there are a number of means to design the GKAs of embodiments of the present invention. These same means may be used to select a candidate compound for screening as a suitable GKA.

For example, there are available computer programs to assist in the process of selecting the agents of certain embodiments of this invention. These include:

1.GRID (Goodford, P. J. A Computational Procedure for Determining Energetically Favorable Binding Sites on Biologically Important Macromolecules. J. Med. Chem., 28, pp. 849-857 (1985)). GRID is available from Molecular Discovery, UK.

2. MCSS (Miranker, A.; Karplus, M.)

Functionality Maps of Binding Sites: A Multiple Copy Simultaneous Search Method. Proteins: Structure. Function and Genetics, 11, pp. 29-34 (1991)). MCSS is available from Molecular Simulations, Burlington, Mass.

3. AUTODOCK (Goodsell, D. S.; Olsen, A. J. Automated Docking of Substrates to Proteins by Simmulated Annealing. PROTEINS: Structure. Function and Genetics, 8, pp. 195-202 (1990)). AUTODOCK is available from the Scripps Research Institute, La Jolla, Calif.

4. DOCK (Kuntz, I. D.; Blaney, J. M.; Oatley, S. J.; Langridge, R.; Ferrin, T. E. A Geometric

Approach to Macromolecule-Ligand Interactions. J. Mol. Biol., 161, pp. 269-288 (1982)). DOCK is available from the University of California, San Francisco, Calif.

Once suitable binding moieties have been selected, they can be assembled into a single compound. This assembly may be accomplished by connecting the various moieties to a central scaffold. The assembly process may, for example, be done by visual inspection followed by manual model building, again using software such as Quanta or Sybyl.

In addition to the above computer assisted modelling of liver Gck expression repressor compounds, the GKAs of certain embodiments may be constructed “de novo” using either an empty active site or optionally including some portions of a known repressor. Such methods are well known in the art. They include, for example:

1. LUDI (Bohm, H. J. The Computer Program LUDI: A New Method for the De Novo Design of Enzyme Inhibitors. J. Comp. Aid. Molec. Design., 6, 61-78 (1992)). LUDI is available from Biosym Technologies, San Diego, Calif.

2. LEGEND (Nishibata, Y., Rai, A., Tetrahedron, 47, 8985 (1991)). LEGEND is available from Molecular Simultations, Burlington, Mass.

3. LeapFrog (available from Tripos associates, St. Louis, Mo.).

4. SPROUT (V. Gillet et al., SPROUT: A Program for Structure Generation)”, J. Comput. Aided Mol. Design, 7, pp. 127-153 (1993)). SPROUT is available from the University of Leeds, UK.

A number of techniques commonly used for modelling drugs may be employed (For a review, see: Cohen, N. C.; Blaney, J. M.; Humblet, C.; Gund, P.; Barry, D. C., “Molecular Modeling Software and Methods for Medicinal Chemistry”, J. Med. Chem., 33, pp. 883-894 (1990)). There are likewise a number of examples in the chemical literature of techniques that can be applied to specific drug design projects. For a review, see: Navia, M. A. and Murcko, M. A., “The Use of Structural Information in Drug Design”, Current Opinions in Structural Biology, 2, pp. 202-210 (1992).

A variety of conventional techniques may be used to carry out each of the above evaluations as well as the evaluations necessary in screening a candidate compound for Gck mRNA expression repression. Generally, these techniques involve determining the location and binding proximity of a given moiety, the occupied space of a bound compound, the deformation energy of binding of a given compound and electrostatic interaction energies. Examples of conventional techniques useful in the above evaluations include: quantum mechanics, molecular mechanics, molecular dynamics, Monte Carlo sampling, systematic searches and distance geometry methods (G. R. Marshall, Ann. Rev. Pharmacol. Toxicol., 27, p. 193 (1987)). Specific computer software has been developed for use in carrying out these methods. Examples of programs designed for such uses include: Gaussian 92, revision E.2 (M. J. Frisch, Gaussian, Inc., Pittsburgh, Pa. ©1993); AMBER, version 4.0 (P. A. Kollman, University of California at San Francisco, ©1993); QUANTA/CHARMM [Molecular Simulations, Inc., Burlington, Mass. 1992]; and Insight II/Discover (Biosysm Technologies Inc., San Diego, Calif. 1992). These programs may be implemented, for instance, using a Silicon Graphics Indigo 2 workstation or IBM RISC/6000 workstation model 550. Other hardware systems and software packages will be known and of evident applicability to those skilled in the art.

The terms “patient”, “subject” and “individual” may be used interchangeably and refer to either a humans or non-human mammal. Aptly, the subject is a human.

The therapeutically effective amount of the GKAs and compositions thereof, as described herein will depend on the route of administration, the type of subject being treated, and the physical characteristics of the specific subject under consideration. These factors and their relationship to determining this amount are well known to skilled practitioners in the medical arts. This amount and the method of administration can be tailored to achieve optimal efficacy, and may depend on such factors as weight, diet, concurrent medication and other factors, well known to those skilled in the medical arts. The GKAs and compositions thereof of the present disclosure may be particularly useful for treatment of humans.

As used herein an “effective” amount or a “therapeutically effective amount” of a GKA refers to a nontoxic but sufficient amount of the GKA to provide the desired effect. The amount that is “effective” will vary from subject to subject, depending on the age and general condition of the individual, mode of administration, and the like. An appropriate “effective” amount in any individual case may be determined by one of ordinary skill in the art using routine experimentation.

An effective dosage and treatment protocol may be determined by conventional means, starting with a low dose in laboratory animals and then increasing the dosage while monitoring the effects, and systematically varying the dosage regimen as well. Numerous factors may be taken into consideration by a clinician when determining an optimal dosage for a given subject. Such considerations are known to the person skilled in the art.

The term “pharmaceutically acceptable carrier” includes any of the standard pharmaceutical carriers. Pharmaceutically acceptable carriers for therapeutic use are well known in the pharmaceutical art, and are described, for example, in Remington's Pharmaceutical Sciences, Mack Publishing Co. (A.R.Gennaro edit. 1985). For example, sterile saline and phosphate-buffered saline at slightly acidic or physiological pH may be used. pH buffering agents may be phosphate, citrate, acetate, tris/hydroxymethyl)aminomethane (TRIS), N-Tris(hydroxymethyl)methyl-3-aminopropanesulphonic acid (TAPS), ammonium bicarbonate, diethanolamine, histidine, arginine, lysine, or acetate or mixtures thereof. The term further encompasses any agents listed in the US Pharmacopeia for use in animals, including humans.

“Treatment” is an approach for obtaining beneficial or desired clinical results. For the purposes of the present disclosure, beneficial or desired clinical results include, but are not limited to, alleviation of symptoms, diminishment of extent of disease, stabilized (i.e. not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. “Treatment” is an intervention performed with the intention of preventing the development or altering the pathology of a disorder. Accordingly, “treatment” refers to both therapeutic treatment and prophylactic or preventative measures in certain embodiments. Those in need of treatment include those already with the disorder as well as those in which the disorder is to be prevented. By treatment is meant inhibiting or reducing an increase in pathology or symptoms when compared to the absence of treatment, and is not necessarily meant to imply complete cessation of the relevant condition.

The term “pharmaceutically acceptable salt” refers to a salt of any one of the GKAs of embodiments of the invention. Salts include pharmaceutically acceptable salts such as acid addition salts and basic salts. Examples of acid addition salts include hydrochloride salts, citrate salts and acetate salts. Examples of basis salts include salts where the cation is selected from alkali metals, such as sodium and potassium, alkaline earth metals, such as calcium, and ammonium ions ⁺N(R³)₃(R⁴), where R³ and R⁴ independently designates optionally substituted C₁₋₆-alkyl, optionally substituted C₂₋₆-alkenyl, optionally substituted aryl, or optionally substituted heteroaryl. Other examples of pharmaceutically acceptable salts are described in “Remington's Pharmaceutical Sciences”, 17^{th} edition. Ed. Alfonoso R. Gennaro (Ed.), Mark Publishing Company, Easton, Pa., U.S.A., 1985 and more recent editions, and in the Encyclopaedia of Pharmaceutical Technology.

Throughout the description and claims of this specification, the words “comprise” and “contain” and variations of them mean “including but not limited to” and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any foregoing embodiments. The invention extends to any novel one, or novel combination, of the features disclosed in this specification (including any accompanying claims, abstract and drawings), or to any novel one, or any novel combination, of the steps of any method or process so disclosed.

Units, prefixes and symbols are denoted in their Système international d'unités (SI) accepted form. Numeric ranges are inclusive of the numbers defining the range. Unless other indicated, amino acid sequences are written left to right in amino to carboxy orientation. All amino acid residues in peptides of embodiments of the invention are preferably of the L-configuration. However, D-configuration amino acids may also be present.

The reader's attention is directed to all papers and documents which are filed concurrently with or previous to this specification in connection with this application and which are open to public inspection with this specification, and the contents of all such papers and documents are incorporated herein by reference.

### EXAMPLES

### Example 1

Materials and Methods

Hepatocyte Isolation and Culture

Hepatocytes were isolated from male Wistar rats (body wt 200-300 g) (Envigo, Bicester, UK). Procedures conformed to Home Office Regulations. The hepatocytes were suspended in Minimum Essential Medium (MEM) 21430 (Life Technologies, GIBCO BRL, Grand Island, N.Y.) with 5% (v/v) calf serum (Life Technologies, GIBCO BRL, Grand Island, N.Y.) and seeded on gelatin-coated plates. After cell attachment (˜4 hours) the medium was replaced by serum-free medium containing 5 mM glucose, 10 nM dexamethasone, 1 nM insulin and experiments were started after ˜20 hours culture by challenge with media containing 5 mM glucose, 15 mM glucose or 25 mM glucose and other additions as indicated.

Hepatocyte Incubations

Parallel incubations were performed for RNA extraction and metabolite determination.

Hepatocytes were incubated for 1 or 2 hours in MEM containing 10 nM insulin and 5, 15, or 25 mM glucose with or without 10μM GKA 3-cyclopentyl-2-(4-methanesulfonylphenyl)-N-thiazol-2-yl-propionamide (RO 28-1675) (Axon MedChem BV, Groningen) for determination of glucose phosphorylation (nmol/2 h per mg) and cell G6P (nmol/mg protein).

Incubations for mRNA analysis and fructose 2,6-bisphosphate were for 4 hrs in MEM with 5, 15, or 25 mM glucose with or without 10 nM insulin and with or without 2 mM fructose. In samples including GKA, 10 μM GKA RO 28-1675 was added.

Incubations with 2-deoxyglucose (DG) where carried out as described previously but with 5 or 10 mM DG with 5 mM glucose (5G) and with or without 2 μM S4048.

Metabolite Determination

Medium containing the cultured hepatocytes was drained from cell culture plates and the plates were snap-frozen in liquid nitrogen and stored at −80° C. For H6P determination, cells were extracted in 2.5% sulphosalicylic acid for deproteinization (10,000g, 10 min) and the supernatants were then neutralized with KOH/KH²PO₄ (3 M/1 M) and metabolites assayed on the neutralized deproteinized extracts. For fructose 2,6-P₂, cells were extracted in 0.15 M NaOH.

For G6P and H6P determination the assay procedure involved coupling of the dehydrogenation of G6P by glucose-6-phosphate dehydrogenase to the dehydrogenation of NADPH (by NADP dehydrogenase) by the reduction of the molecule resazurin to resorufin by NADP measured by the absorbance of resorufin using a spectrophotometer. Standards of G6P (2 to 20 μM) were prepared in 2.5% sulphosalicylic acid and neutralized with KOH/KH₂PO₄ (3 M/1 M) as in samples for metabolite determination. Standards and samples (20 μl) were then assayed in a final reaction volume of 200 μI containing 50 mM triethanolamine (TEA, pH 7.6), 1.0 mM MgCl2, 100 μM NADP+, 10 μM resazurin, 0.1 U/ml G6PD, and 0.2 U/ml diaphorase. The mixtures were then incubated for 30 min at room temperature. Fluorescence at 590 nm was measured using excitation at 530 nm using a plate reader (M5E Molecular devices). Background fluorescence was corrected by subtracting the value of the no-G6P control from all sample readings and sample values were read off the linear standard curve determined from the G6P standards.

Fructose 2,6-bisphosphate was determined from the activation of pyrophosphate: fructose 6-phosphate phosphotransferase from potato tuber (PPi-PFK) in a kinetic assay in a final assay mixture containing: 50 mM Tris/acetate; 2 mM Mg-acetate, 1 mM fructose 6-P, 0.15 mM NADH, 10 mU/ml PPi-PFK; 0.45 U/ml aldolase; 5 units/ ml triose phosphate isomerase; 1.7 Units/ml glycerol phosphate dehydrogenase; 0.5 mM tetrasodium pyrophosphate (all reagents were from Sigma-Aldrich). The reaction was monitored from the decrease in absorbance of NADH at 340 nm in a M5E plate reader (Molecular Devices). A standard curve of 5-300 nM fructose 2,6-bisphosphate was used. The volume of sample or standard was 5 μl or 10 μl in a final reagent volume of 100 μl in a 96-well plate. Sample fructose 2,6-bisphosphate concentration was determined from the standard curve which was sigmoidal.

Metabolites are expressed as nmol/mg protein except for fructose 2,6-P2 which is expressed as pmol/mg of total cell protein determined by a Lowry assay.

Glucose Phosphorylation Determination

Medium contained [2-³H] glucose (1 μCi/ml ). Glucose phosphorylation was determined from the formation of ³H₂O. Hepatocytes were incubated in MEM containing glucose (5, 15, 25 mM) and 1 μCi/ml of [2-³H] glucose. On termination of incubation the medium was acidified with 0.1 M HCl and an aliquot (50 μl) of acidified medium was equilibrated in an ependorf tube within a sealed container with 750 μl water. After 2 days of equilibration the radioactivity of the water was determined. Formation of ³H20 from [2-³H] glucose is expressed as nmol of glucose metabolised per hour per mg cell protein. [2-³H] glucose was from Perkin Elmer.

mRNA Analysis

RNA was extracted using TRIzol (Invitrogen) and cDNA was synthesized from 1 μg of RNA with MMLV (Promega). Real-time RT-PCR was performed in a total volume of 10 μl containing 50 ng of reverse transcribed RNA and 5 ng of forward and reverse primers using a Sybr-Green protocol (Promega) in a Roche Capillary Light Cycler. The primers were as shown in Table 1. Relative mRNA levels (vs control) were calculated by the delta cycle threshold method.

TABLE 1
Primers for mRNA analysis

| Target | Forward | Reverse | Gene |
|---|---|---|---|
| Gene | Primer | Primer | identity |
| Gck | TTGCTCTAAGGG | GGAACGAGGGAG | NM_012565 |
| | GACCAGAA | AGAAGGAC | (383-550) |
| | (SEQ ID NO: 1) | (SEQ ID NO: 2) | |
| G6pc | CTACCTTGCGGC | ATCCAAGTGCGA | NM_013098 |
| | TCACTTTC | AACCAAAC | (579-733) |
| | (SEQ ID NO: 3) | (SEQ ID NO: 4) | |
| Pklr | CTGGAACACCTC | CACAATTTCCAC | NM_012624 |
| | TGCCTTCTG | CTCCGACTC | (130-456) |
| | (SEQ ID NO: 5) | (SEQ ID NO: 6) | |

Statistical Analysis

Data are means±Standard Error of Mean (SEM) for the number of hepatocyte preparations indicated. Statistical analysis was by Student's t-test (paired or unpaired). Correlations were by linear regression.

Results

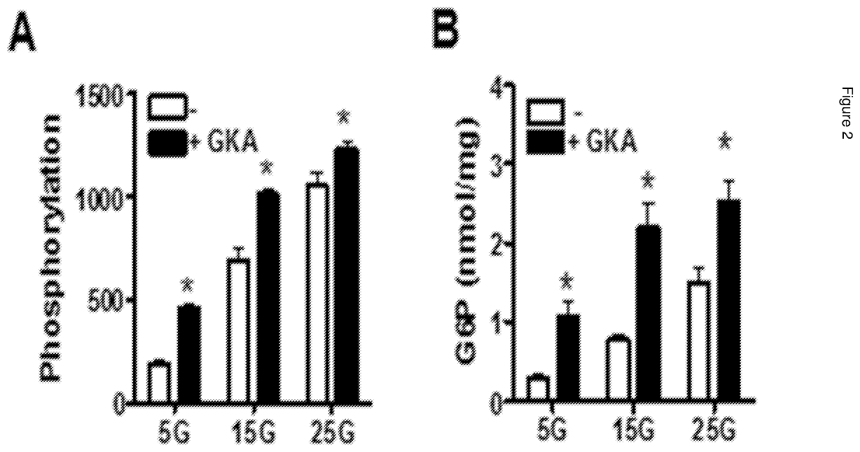

Referring to FIGS. 2A and 2B the effects of the GKA at different glucose concentrations is shown. The GKA increases the rate of phosphorylation of glucose (metabolic flux) at all glucose concentrations (2A) and increases the cell concentration of G6P at all glucose concentrations (2B). The effect of the GKA on the fractional increase in G6P at basal (5 mM) and moderate (15 mM) glucose is greater than the fractional increase in metabolic flux.

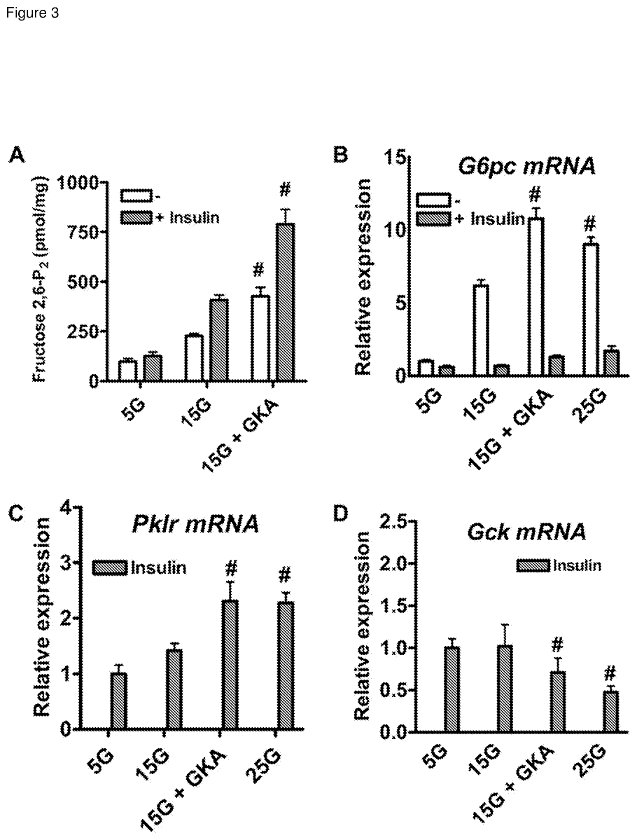

Without being bound by theory this raise in G6P is believed to provide positive stimulation of phosphofructokinase 2/fructose-2,6-bisphosphatase (PFKFB2) which increases the conversion of F6P to Fructose-2,6-bisphosphate (F-2,6-P₂). This increase in F-2,6-P₂ in response to the GKA is shown by the graph in FIG. 3A.

Without being bound by theory, F-2,6-P₂ is believed to act as an activator of Max like proteins (Mix) binding partners such as Mondo-A and the Carbohydrate Responsive Element Binding Protein (ChREBP). The MIx:ChREBP complex is an inducer of the G6pc and Pklr genes and MIx in conjunction with other partners is also a repressor of the GK coding gene Gck. The stimulation of MIx target genes by the GKA is shown by the increase in relative expression of both Pklr and G6pc mRNA when cells are incubated with the GKA (FIG. 3B and 3C) and also the decrease in relative expression of Gck mRNA when cells are incubated with the GKA (FIG. 3D).

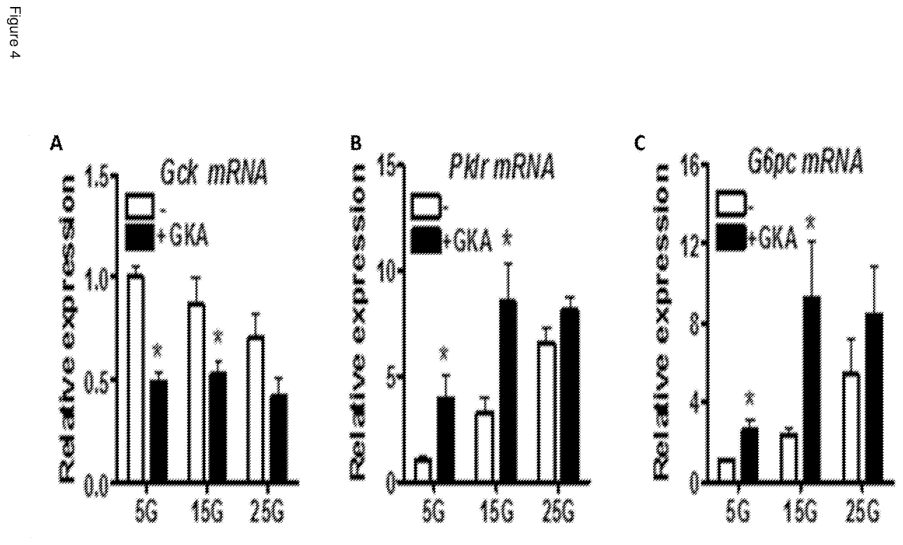

It can be seen from the graphs shown in FIG. 4 that the GKA represses the relative expression of Gck at basal (5 mM) glucose, medium (15 mM) glucose (as after an intake of food) and high (25 mM) glucose (FIG. 4A). It can also be seen that the GKA induces the expression of both G6pc and Pklr mRNA as is shown by the increase in relative expression of these two mRNAs at all glucose concentrations when the GKA is present (FIGS. 4B and 4C).

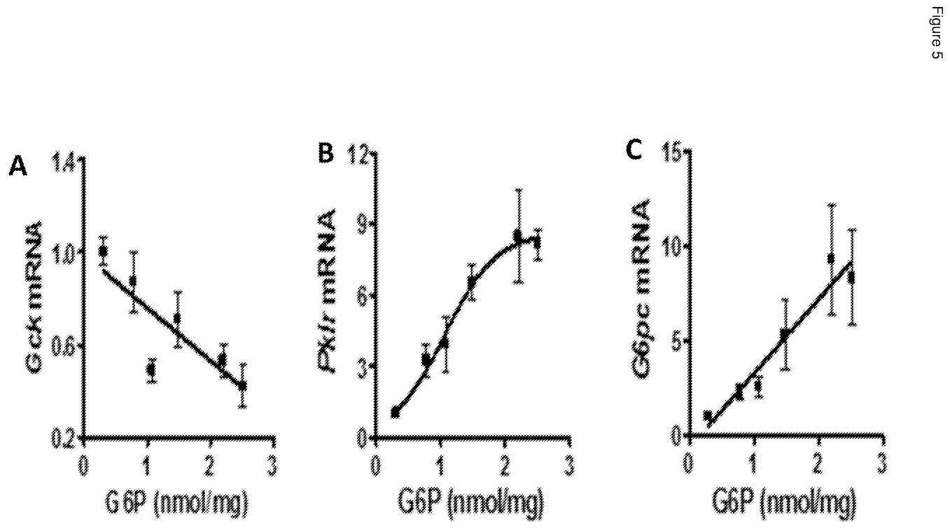

The decrease in the relative expression of Gck mRNA can be seen to be negatively correlated with cell G6P concentration (FIG. 5A), indicating that an increase in G6P leads to a decrease in Gck mRNA and therefore lower GK concentrations which in turn may lead to lower GK activity. The graphs shown in FIG. 5B and 5C also show that relative expression of Pklr and G6pc mRNA is positively correlated with G6P concentration within the cell.

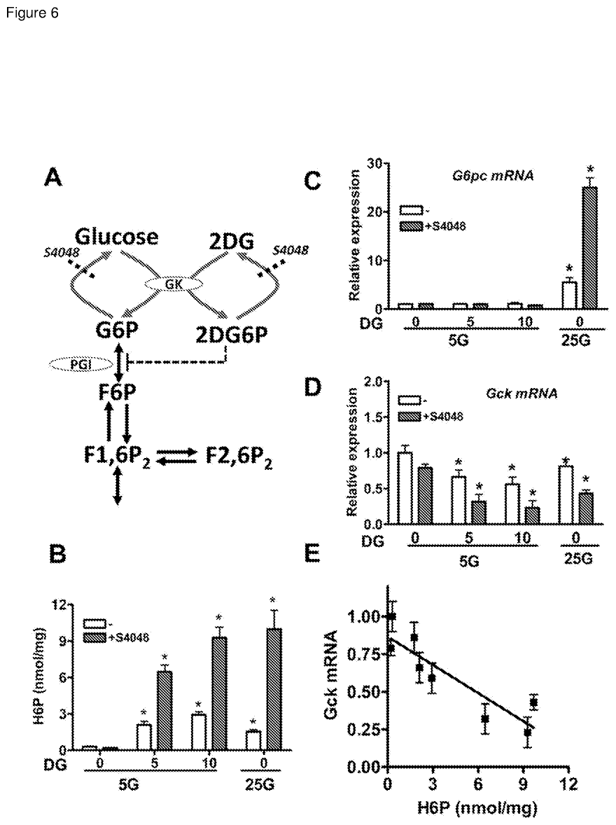

It can be seen from the diagram shown in FIG. 6A that when hepatocytes are incubated with the glucose analogue 2-deoxyglucose (DG), DG is phosphorylated by liver GK in hepatocytes to the corresponding hexose 6-phosphate, 2-deoxyglucose 6-phosphate, an analogue of glucose 6-phosphate. 2-deoxyglucose 6-phosphate (unlike glucose 6-phosphate) cannot be further metabolised to fructose 6-phosphate (F6P) or fructose 2-6-bisphosphate (F2,6P2). The agent S4048 inhibits transport and hydrolysis of G6P and 2-deoxyglucose 6-phosphate to glucose and DG, therefore increasing the level of H6P. The DG causes elevation in the cell hexose 6-phosphate (H6P) content (FIG. 6B) and repression of Gck mRNA (FIG. 6D) but it does not cause induction of expression of G6pc mRNA (FIG. 6C) which is a target gene of Mix-Oh REBP. Not being bound by theory, the data indicates that the hexose 6-phosphate products of the liver GK conversion of glucose and/or DG to G6P and 2-deoxyglucose 6-phosphate causes repression of the Gck gene but does not causes induction of target genes of Mix-Oh REBP like G6pc. Thus the data shows that H6Ps alone are able to repress the Gck gene. The expression of Gck mRNA correlated negatively with the H6P concentration (FIG. 6E).

In summary the data shows that the GKA is able to reduce Gck expression. Not being bound by theory the data further indicates that this most likely occurs by the GKA increasing the levels of G6P.

### Example 2

Example 2 relates testing of a GKA and its effects on animal and human NAFLD and NASH development and treatment.

Titration with a GKA to Test for Hepatic Compensation

An acute exposure titration is used to determine the dose of the GKA for liver Gck mRNA repression. Groups of C57BL/6 male mice (n=12/group) are fasted overnight and given an oral load of pharmaceutically acceptable diluent or excipient (e.g. 1% Pluronic F127), a control GKA, e.g. (S)-6-(3-Cyclopentyl-2-(4-(trifluoromethyl)-1H-imidazol-1-yl)propanamido)nicotinic Acid (PF-04995132) (100mg/kg) or the GKA to be tested (0.5, 1, 2 or 5 mg/kg) and after 1 hour a glucose tolerance test (GTT) (2g/kg) is performed for analysis of glucose and insulin at −60, 0, 15, 30, 60, 120 min. Mice are euthanized for liver removal and mRNA (Gck, G6pc and Pklr), GK activity and G6P analysis. Based on a standard deviation of 45% of the mean, n=12 enables detection at 0.8 power, P<0.05 of 40% difference in Gck mRNA.

Chronic Study for Hepatic Adaptations Over 4 to 8 Weeks

Groups of C57BL/6 male mice (n=18/group) are fed chow −/+ a GKA. A GTT is performed at day 1, 4 weeks and 8 weeks (for glucose and insulin). Blood is collected every 7 days for glucose, insulin and TAG analysis. For each group: 8 mice are euthanized at 4 weeks and 10 mice at 8 weeks for GK and GKRP immunoactivity, lipid extraction and hepatocyte isolation (n=8) for the following endpoints: (i) the affinity for glucose and rates of phosphorylation, glycolysis, lipogenesis, glycogenesis using [2-³H]glucose, [3-³H]glucose, [U-¹⁴C]glucose and the partitioning of glucose metabolic flux between alternative pathways determined by [1,2-¹³C2] glucose and mass isotopomer distribution analysis; (ii) the effect of glucose (25 mM)−/+insulin challenge on cell G6P (10-120 min time course) and mRNA expression of glucose-regulated genes (Gck, Gckr, G6pc and Pklr).

Liver GK And GKRP Immunohistochemistry in Human NAFLD

Tissue blocks from up to 100 NAFLD biopsies are processed for: (i) GK and GKRP immunohistochemistry (IHC); (ii) histopathology scores of steatosis, lobular/periportal inflammation, hepatocyte ballooning and fibrosis. This is correlated with (iii) CHIP based mRNA expression for Gck, Gckr and glucose-regulated genes (e.g Pklr); (iv) genotyping for gene variants linked to NAFLD.

The following are then correlated: (i) GK and GKRP immunostaining topography (by digital scanning and semi-quantitative imaging) with histopathology, to determine whether GK immunohistochemical expression correlates with steatosis as predicted; (ii) GK staining intensity with Gck mRNA; (iii) GK staining intensity and mRNA expression of glucose-inducible genes to test for a potential causative role of GK in steatosis; (iv) GK and GKRP intensity with Gckr genotype to determine whether a minor Gckr allele associates with altered GK or GKRP protein. This data informs on the degree of concordance between mouse models and human NAFLD.

Effects of a GKA on NAFLD Development in Wild-Type and GKRP-Deficient Mice

GKRP_{+/-} mice on C57BL/6 background are bred to generate GKRP_{+/+} and GKRP_{+/-} (n=40) and at age 6 weeks, are fed on a trans-fat diet (45%:40% fat: carbohydrate +4% glucose) for 4 weeks and then are randomized based on body weight to 2 groups (n=20) that a receive diet −/+ a GKA for 12 weeks. A GTT is performed at 4 weeks, 8 weeks and 12 weeks and an insulin tolerance test (ITT: 1.5 units/kg human insulin, i.p and blood sampled at 0, 30, 60, 90 120 min for glucose) at 6 weeks. Blood is collected at 7 day intervals for glucose, insulin, TAG and alanine aminotransferase analysis. Mice are euthanized at 12 weeks for liver fixation for histopathology, lipid extraction, immunohistochemistry (IHC) and immunoactivity (n=12) and for hepatocyte isolation and functional analysis (n=8). For histology, fragments of the left lateral and right medial liver lobes are formalin-fixed, paraffin-embedded, sectioned and stained with haematoxylin & eosin, Sirius red Fast green and Masson's trichome, CK8+18, CD68, GK, GKRP. They are scored for micro and macrovesicular steatosis, ballooning, lobular/portal inflammation and fibrosis by an expert liver pathologist blinded to the study. Immunostaining is quantified by digital, semi-quantitative analysis.

1-10. (canceled)

11. A method of treating and/or preventing a liver disease comprising administering to a subject in need thereof a therapeutically effective amount of a Glucokinase activator (GKA) or a GK ligand or a pharmaceutically acceptable salt or derivative thereof or a pharmaceutical composition comprising the GKA or a GK ligand and one or more pharmaceutically acceptable carriers.

12. The method according to claim 11, whereby the GKA or GK ligand is administered for a period of time greater than four weeks.

13. (canceled)

14. A method of increasing or modulating glucose-6-phosphate and/or fructose-2,6-bisphosphate concentration in a subject, comprising administering to the subject a GKA or a GK ligand or a pharmaceutically acceptable salt or derivative thereof or a pharmaceutical composition comprising the GKA and one or more pharmaceutically acceptable carriers.

15. The method according to claim 14, whereby the GKA or GK ligand is administered for a period of time greater than four weeks.

16. The method according to claim 14, further comprising administering at least one further therapeutic agent.

17. The method according to claim 14, wherein the GKA is 3-{[5-(azetidin-1-carbonyl)pyrazin-2-yl] oxy}-5-{[(1S)-1-methyl-2-(methyloxy)ethyl] oxy}-N-(5-methylpyrazin-2-yl)benzamide or a pharmaceutically acceptable salt or derivative thereof.

18. A method of reducing GKA activity in a subject, comprising administering to the subject a GKA or other GK ligand or a pharmaceutically acceptable salt or derivative thereof or a pharmaceutical composition comprising the GKA or other GK ligand and one or more pharmaceutically acceptable carriers.

19. The method according to claim 18, whereby the GKA is administered for a period of time greater than four weeks.

20. The method according to claim 18, further comprising administering at least one further therapeutic agent.

21. The method according to claim 18, wherein the GKA is 3{[5-(azetidin-1-carbonyl)pyrazin-2-yl]oxy}-5-{[(1S)-1-methyl-2-(methyloxy)ethyl]oxy}-N-(5-methylpyrazin-2-yl)benzamide or a pharmaceutically acceptable salt or derivative thereof.

22. (canceled)

23. A method of treating and/or preventing steatosis in a subject in need thereof, comprising administering to the subject a GKA or a GK ligand or a pharmaceutical composition comprising the GKA or a GK ligand and one or more pharmaceutically acceptable carriers.

24. The method according to claim 23, whereby the GKA or a GK ligand is administered for a period of time greater than four weeks.

25. The method according to claim 23, further comprising administering at least one further therapeutic agent.

26. The method according to claim 23, wherein the GKA is 3-{[5-(azetidin-1-carbonyl)pyrazin-2-yl]oxy}-5-{[(1S)-1-methyl-2-(methyloxy)ethyl]oxy}-N-(5-methylpyrazin-2-yl)benzamide or a pharmaceutically acceptable salt or derivative thereof.

27. The method according to claim 23, wherein the steatosis is hepatic steatosis.

28. (canceled)

29. The method according to claim 11, wherein the liver disease is a fatty liver disease.

30. The method according to claim 29, wherein the fatty liver disease is selected from one or more of Non-alcoholic Fatty Liver Disease (NAFLD) and/or Non-alcoholic Steatohepatitis (NASH).

31. The method according to claim 11, wherein the GKA or GK ligand is relatively liver selective.

32. The method according to claim 11, wherein the GKA is 3-{[5-(azetidin-1-carbonyl)pyrazin-2-yl]oxy}-5-{[(1S)-1-methyl-2-(methyloxy)ethyl]oxy}-N-(5-methylpyrazin-2-yl)benzamide or a pharmaceutically acceptable salt or derivative thereof.

33. The method according to claim 11, which comprises administering at least one further therapeutic agent to the subject.

34. The method according to claim 33, which is for the treatment of a subject suffering from a liver disease and type II diabetes and/or metabolic syndrome.
